(19) United States
(12) Patent Application Publication
(10) Pub. No.: US 2025/0050043 A1
(43) Pub. Date: Feb. 13, 2025

(54) BREATHING ASSISTANCE APPARATUS WITH CUEING

(71) Applicant: Just Breathe LLC, Lincoln, RI (US)

(72) Inventors: Gregory Calderiso, Lincoln, RI (US); Zachary Bradford Konsin, Cranston, RI (US)

(73) Assignee: Just Breathe LLC, Lincoln, RI (US)

(21) Appl. No.: 18/794,756

(22) Filed: Aug. 5, 2024

Related U.S. Application Data
(60) Provisional application No. 63/518,682, filed on Aug. 10, 2023.

(51) Int. Cl. A61M 16/00; A61M 21/00; A61M 21/02

(52) U.S. Cl. CPC A61M 16/0051; A61M 16/0057; A61M 16/022; A61M 21/02; A61M 2021/0022

(57) ABSTRACT

The present invention includes a device for assisting cued breathing that includes a housing with a two-way air passage to allow two-way passage of breath air to and from the user's mouth. A first nozzle is located in a mouth port to provide haptic air flow into the user's mouth and a second nozzle located on the housing of the apparatus to provide haptic cueing to another portion of the user's body, such as the face, lips, and chin, to assist a user's breathing cadence. The device assists the user to maintain a steady cadence to help reduce stress, anxiety and other ailments.

### CROSS REFERENCE TO RELATED APPLICATION

This patent document claims priority to earlier filed U.S. Provisional Patent Application Ser. No. 63/518,682, filed Aug. 10, 2023, the entire contents of which are incorporated herein by reference.

### BACKGROUND OF THE INVENTION

The invention relates to the field of breathing assistance devices, namely breathing assistance devices that assist users with staying on a proper breathing cadence with proper form and direction of breathing, namely, inhaling and exhaling.

During the course of the day, a person's breathing may become unstable or improper where the cadence of their breathing and respiration rate is abnormal. Or, for example, a person may experience anxiety, stress or other mental concern and be desirous of carrying out breathing exercises, otherwise known as “guided breath work” or “mindful breathing” which have been known to help with such anxiety, stress, or other similar events. Therefore, proper breathing exercises have been clinically proven to increase heart rate variability (HRV), lower heart rate, lower blood pressure, and reduce the level of stress and anxiety experienced by a person.

However, while controlled breathing is known to assist with these ailments, it is important proper breathing techniques be practiced for optimal results.

For example, it is known in medicine that a good breathing technique can greatly assist a person's control of their respiration rate and rhythm and thereby reduce stress, anxiety and the like. There are known attempts to provide such breathing whereby the person executes the breathing exercises on their own in an unassisted manner. For example, the person can simply breathe in, such as through their nose, pause, and then thereafter, for example, breathe out through their mouth. Also, it is common and well-known for a person to count in their head up to a certain number while they are inhaling through their nose (such as three seconds), count to a certain number while they are pausing/holding their breath (such as 2 seconds), and then also count in their head while they are exhaling through their mouth (such as four seconds). All of such counting is intended to develop a cadence of breathing that is repeatable and consistent for an optimal calming effect.

However, such self-driven breathing exercises and techniques rely on a person's focus and concentration as well as accurate counting to ensure consistent and repeatable breathing patterns. In addition, a person must be taught the techniques. It is possible that a person might not remember the proper technique or execute it improperly or inconsistently, which results in less than optimal calming and therapeutic results. This is common and not surprising because such exercises are intended to promote a calming and soothing effect which can result in the person not staying focused on the cadence of the breathing exercise. Thus, the strict manual counting and tracking of breathing conflicts with the desired result of the person being more relaxed.

Also, there are pharmaceutical, psychotherapy, and meditation applications available, such as Calm, to manage stress and anxiety. As far as wearable technology, there have been many attempts to help people manage stress and anxiety. For example, there are monitoring devices that have the ability to instill a state of calm and some do offer breathing options. However, these devices are typically reactive, providing feedback after recognizing stress symptoms rather than actively guiding breathwork to prevent the onset of these symptoms. None of the devices offer guided breathwork through airflow cueing.

For example, there are also breathing sensors in the prior art that are placed on the upper abdomen that automatically analyzes a person's breathing pattern and creates a personalized melody composed of two distinct inhale and exhale guiding tones. It is defined by some medical experts as a bio-feedback device. Similarly, there are respiratory trainers that are generally geared towards enhancing athletic performance or improving specific health conditions rather than preventing or managing anxiety symptoms. They are typically done with resistance-based training.

Firstly, screen-based cues require an individual's visual focus, which could be disruptive to the process of relaxation and concentration that mindful breathing exercises aim to induce. Mindful breathing involves internal focus and a sense of disconnection from external stimuli; constant visual engagement with a screen contradicts this principle.

Secondly, the exposure to screen light, particularly in low-light settings or before sleep, can negatively impact the user's circadian rhythm, sleep quality, and overall relaxation. The blue light emitted from screens has been scientifically proven to interfere with the production of melatonin, the hormone that regulates sleep-wake cycles.

Lastly, a screen-based breathing aid demands the user's undivided attention and restricts their mobility. It limits the contexts in which the user can perform the breathing exercises, potentially inhibiting consistent practice and adherence to the mindful breathing regimen.

For these reasons, an alternative method of delivering breathing cadence and cues, one that doesn't rely on screen interaction, is both beneficial and necessary.

In view of the common difficulty in staying focused on these breathing techniques, there is a need for a device, method and system that assists the person in keeping the proper cadence without the person having to closely focus on the cadence of the breathing.

In the prior art, there have been attempts to address the foregoing concerns by providing various types of devices through which the user breathes. For example, such a prior art device might include a housing that the person places their lips on while breathing to accentuate the actions of exhaling to provide a calming air flow sound during the breathing exercise. However, these devices are devoid of any cueing or guidance to help the user keep a consistent and proper cadence. In these devices, the cadence and cueing for each of the steps of a proper breathing exercise is up to the person to execute.

In view of the above, there is a need for a device that not only physically directs airflow but also provides important guidance and cueing to keep the person on a proper cadence thereby allowing the person to focus on the breathing itself and the benefits therefrom rather than on constantly counting or looking at a computer or mobile device screen.

There is a further need for such an apparatus to provide a mechanism for cueing the user consistently and accurately.

There is a need for a portable device to assist with breathing exercise cadence.

There is a need for a non-invasive device to assist with breathing exercise cadence.

There is another need for an apparatus that is easy to use and inexpensive in cost.

A further needs is for an apparatus that is easy, simple and inexpensive to manufacture.

### SUMMARY OF THE INVENTION

The invention provides an improved device for assisting a user in breathing at a steady cadence for various well-being purposes, including the reduction of stress and anxiety, and other health concerns that benefit from calming breathing exercises.

The device of the present invention provides a non-pharmacological approach to managing mental health, stress, and anxiety by utilizing breath work which the user is guided through by airflow cues anywhere on or near the user, such as into the mouth, into the nose, on the face, or the lips, for example. The air flow cues signal the user when to inhale and exhale, along with pre-selected or app-recommended patterns that are clinically proven to induce a state of calm in the nervous system.

The device may also provide vibrational haptic feedback alongside machine learning algorithms to learn the user's specific physiology and make recommendations for breath work patterns to in turn provide the airflow cues. The device of the present invention helps a person with the treatment of mental health and wellness while, for example, catering to individuals who suffer from anxiety, stress, depression, and other related disorders. Furthermore, it has potential applications in the realm of Integrative and Complementary Health approaches for pain management.

By leveraging the power of breathwork, the device of the present invention has wide-ranging implications for health and wellness, mental health, stress management, and mind-body interventions in general. This not only applies to clinical or therapeutic settings but also in day-to-day personal health management and mindfulness practice.

More specifically, the present invention can be implemented in various health and wellness scenarios, including personal daily stress management, professional mental health therapy as an adjunct tool, workplace wellness programs, and therapeutic contexts like Cognitive Behavioral Therapy (CBT) or mindfulness-based therapy. The device could also be used in professional settings, including therapy practices, wellness clinics, rehabilitation centers, and hospitals, providing healthcare providers with an innovative tool for their patients.

The device of the present invention is handheld, portable, easy to use, inexpensive in cost, and is of a user-friendly design to provide self-managed, non-invasive techniques for improving their mental well-being. The device is also inexpensive to manufacture.

Therefore, an object of the present invention is to provide a breathing assistance apparatus with cueing that specifically assists with the user's cadence of breathing.

A further object of the invention is to physically help guide airflow but also provide important guidance and cueing to keep the person on a proper cadence, thereby allowing the person to focus on the breathing itself and take advantage of the benefits therefrom rather than on constantly counting.

Another object of the present invention is to provide a mechanism for cueing the user consistently and accurately.

Yet another object of the present invention is to provide a portable device to assist with breathing exercise cadence.

Another object of the present invention is to provide a non-invasive device to assist with breathing exercise cadence.

Yet a further object of the present invention is to provide an apparatus that is easy

to use and inexpensive in cost.

A further object of the present invention is to provide an apparatus that is easy, simple and inexpensive to manufacture.

### BRIEF DESCRIPTION OF THE DRAWING FIGURES

The novel features that are characteristic of the present invention are set forth in the appended claims. However, the invention's preferred embodiments, together with further objects and attendant advantages, will be best understood by reference to the following detailed description taken in connection with the accompanying Figures in which:

FIG. 1 is a front perspective view of the breathing assistance apparatus in accordance with the present invention;

FIG. 2 is a rear perspective view of the breathing assistance apparatus of FIG. 1;

FIG. 3 is a front elevational view of the breathing assistance apparatus of FIG. 1;

FIG. 4 is a rear elevational view of the breathing assistance apparatus of FIG. 1;

FIG. 5 is a top view of the breathing assistance apparatus of FIG. 1;

FIG. 6 is a bottom view of the breathing assistance apparatus of FIG. 1;

FIG. 7 is a front perspective cross-sectional view of the breathing assistance apparatus in accordance with the present invention;

FIG. 8 is a side cross-sectional view of the breathing assistance apparatus in accordance with the present invention;

FIG. 9 is a side elevational view of the breathing assistance apparatus in accordance with the present invention showing interconnection of nozzles to the housing using connectors;

FIG. 10 shows a perspective view of a connector used for securing a nozzle to the housing of the present invention;

FIG. 11 shows an exploded perspective view of a nozzle for delivering air to into the housing;

FIG. 12 is a top view of an embodiment of a circuit board with microprocessor, pumps and other controls for use in operating the breathing assistance apparatus of the present invention;

FIG. 13 is a front view of a smartphone showing use of an app for controlling the breathing assistance apparatus of the present invention;

FIG. 14 is a front view of a computer showing use of an application for controlling the breathing assistance apparatus of the present invention; and

FIGS. 15A and 15B show, respectively, graphs of variable heart rate of an individual before and after use of the breathing assistance apparatus of the present invention; and

FIG. 16 shows a partial cross-sectional view of an alternative embodiment of the present invention where all components are integrated into the housing to provide an entirely self-contained breathing assistance apparatus.

### DESCRIPTION OF THE INVENTION

The new and unique breathing assistance apparatus 10 with cueing is shown in detail in the attached figures. The breathing assistance apparatus device 10 of the present invention is non-invasive and non-pharmacological, namely, a non-invasive, drug-free alternative for anxiety management. This makes it a safe choice for individuals who may be concerned about the potential side effects of medications. Since it is a non-pharmacological treatment option that users can administer themselves, the apparatus 10 can potentially lower overall treatment costs compared to traditional therapy or medication.

Moreover, the breathing assistance apparatus 10 of the present invention enables anxiety and stress to be reduced through breathwork that is guided by the device's airflow cueing. The apparatus 10 is portable with a user-friendly interface (buttons on the device with pre-selected patterns and/or mobile application) that can be used anywhere and anytime. This facilitates regular and consistent use, making anxiety management readily accessible to everyone and reducing barriers to treatment.

Turning now to the figures, the construction, configuration, use and companion application of the present invention is shown in detail.

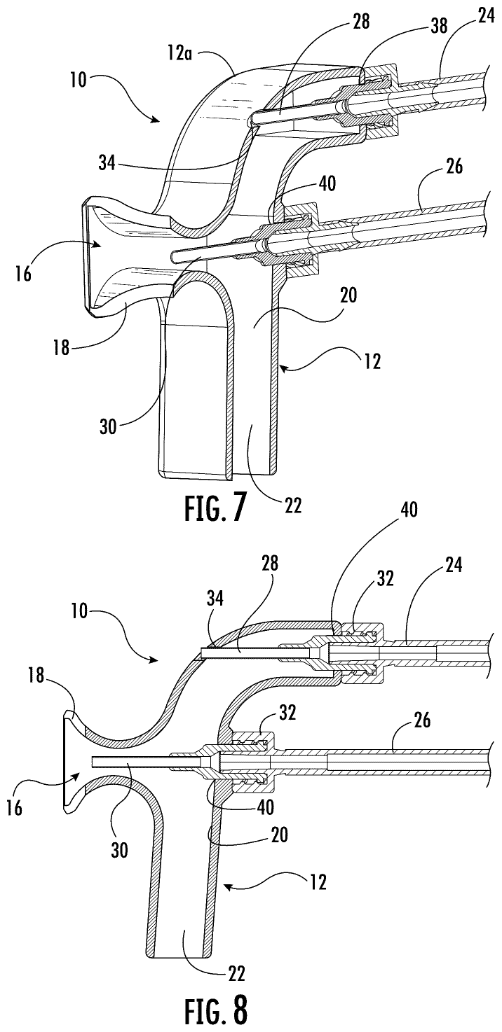

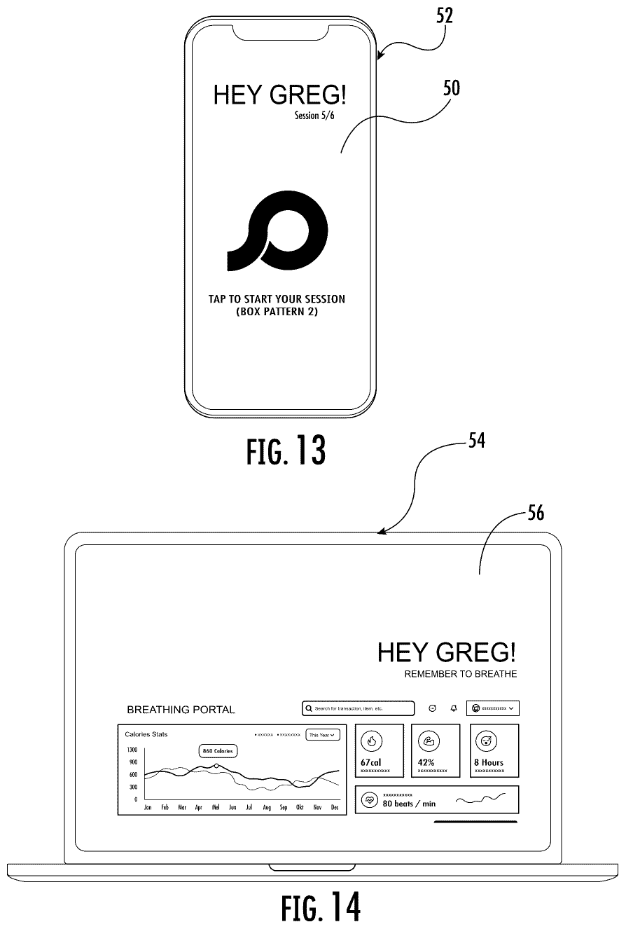

FIGS. 1-6 show various views of the preferred embodiment of the breathing assistance apparatus 10 with cueing of the present invention is shown while FIGS. 7-12 show various components and interior construction of the present invention. FIGS. 13-15 show use of the breathing assistance apparatus 10 of the present invention to reduce stress, anxiety, improve heart rate variability, and provide other physical benefits.

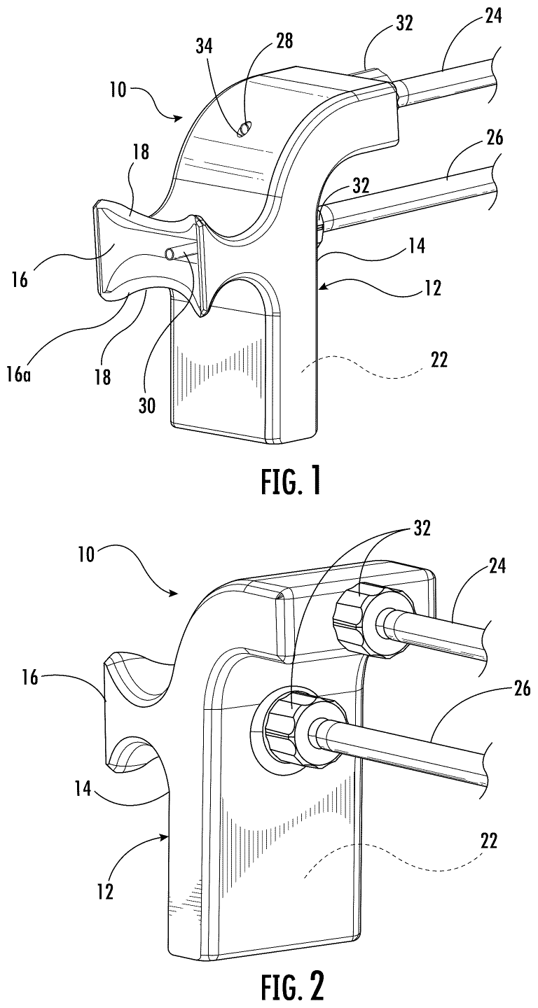

Referring now to FIG. 1, a front perspective view of the breathing assistance apparatus 10 in accordance with the present invention is shown while FIG. 2 shows a rear perspective view thereof. The apparatus 10 includes a housing 12 of a box-like configuration that preferably has a substantially hollow interior 20 with a main body portion 14. A two-way mouth port 16 is provided for receipt of the user's mouth where the user places their mouth on the opening, preferably where their lips communicate with the port 16. The port 16 is configured with curved walls 18 to facilitate communication with a user's mouth. The mouth port preferably extends laterally outward from the main body portion 14.

Extending downwardly from the main body 14 is a pass-through breathing channel 22 whereby air flows freely in two directions and is in fluid communication with the mouth port 16. Thus, the user can breathe freely unimpeded when their mouth is on the mouth port 16 and their lips are fully sealed thereto. Further details of the pass-through breathing channel 22 will discussed in detail below.

As also can be seen in FIG. 1, air is supplied, via two tubes 24, 26, to two different air cueing nozzles 28, 30, one mouth nozzle 30 for the central haptic air flow into the user's mouth via the mouth port 16 and another body nozzle 28 for blowing haptic air to the lip, face, mouth, or any area or body portion of the user. While FIG. 1 shows one nozzle 30 directed into the user's mouth and another nozzle 28 to the user's body, such as their face or lip area, both nozzles 28, 30 can be located and aimed at any part of the user's body and in any combination thereof.

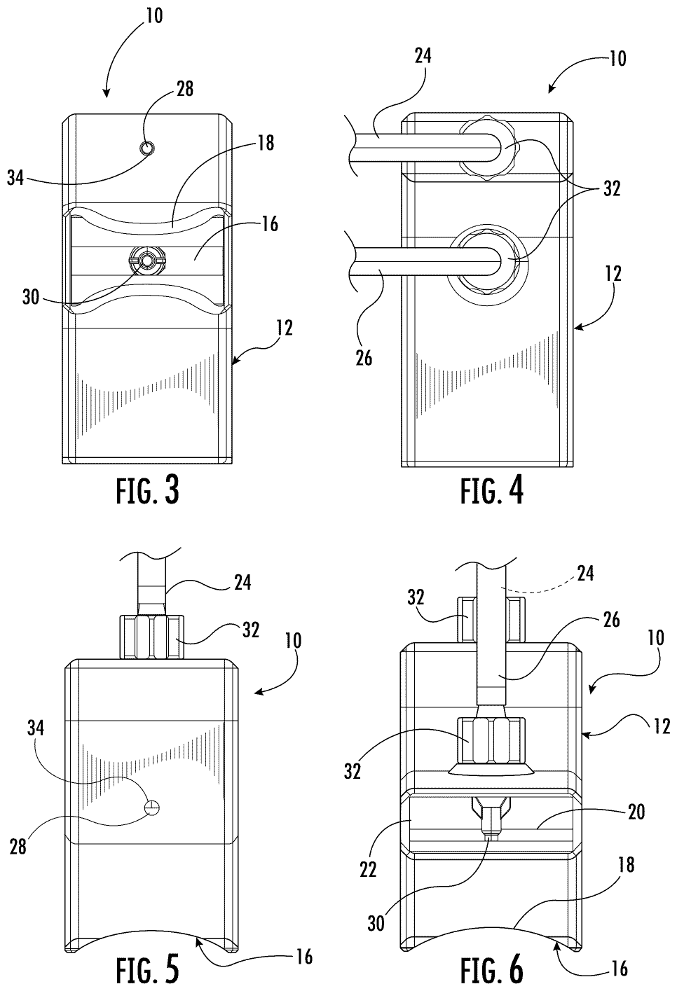

FIG. 3 shows a front elevational view of the breathing assistance apparatus 10 of FIG. 1 where the preferred shape of the mouth port 16 can be clearly seen. The mouth port 16 preferably has curved top and bottom portion 18 but also is laterally curved, as can be seen in the top view of the apparatus 10, as seen in FIG. 5.

FIG. 4 shows a rear elevational view of the breathing assistance apparatus 10 of FIG. 1 where the supply air lines 24, 26 are interconnected to the housing 12 with respective connectors 32. The supply lines 24, 26 are in fluid communication with the respective mouth nozzle 30 and body nozzle 28 residing inside the housing 12 to supply the haptic air in accordance with the present invention. The supply lines 24, 26 are preferably made of a flexible material, such as nylon tubing, but could be any flexible material and sized as desired to meet the needs of the user. Moreover, the air supply lines 24, 26 are shown in FIG. 4 as entering laterally from the left to connected to the housing 12 but that is for illustration purposes only so as much of the rear side of the main body 14 of the housing 12 can be seen.

Referring back to FIG. 5, which is a top view of the breathing assistance apparatus 10 of FIG. 1, haptic air supply line 24 can be seen entering into the rear of the main body 14 of the housing 12 and is secured in place with a connector 32. A top hole 34 is provided through a top portion of the housing 12 which is axially aligned and in fluid connection with the body nozzle 28, as can also be seen in FIGS. 7 and 8. Thus, haptic air is pumped through a top supply line 24, through the body nozzle 28 and then through the top hole 34 to provide haptic air laterally to the face of the user, such as the lip area. As stated above, this hole 34 may be modified to direct haptic air to another location and in a different direction. Referring back to FIG. 3, this top hole 34 can be seen above the mouth port 16. Also in FIG. 3, the mouth nozzle 30 can be seen inside the mouth port 16 to provide haptic air into the user's mouth.

FIG. 6 is a bottom view of the breathing assistance apparatus 10 of FIG. 1 that further shows the two air supply lines 24, 26 that are connected to the housing 12. In this view, the mouth nozzle 30 is visible through the open pass-through breathing channel 22 at the bottom of the apparatus 10. The free end of the mouth nozzle 30 is preferably located proximal the edge 16a of the middle of the mouth port 16.

FIG. 7 shows a front perspective cross-sectional view of the breathing assistance apparatus 10 in accordance with the present invention while FIG. 8 illustrates a side cross-sectional view of the breathing assistance apparatus 10 in accordance with the present invention. The location and positioning of the mouth nozzle 30 and body nozzle 28 through the housing 16 is shown in detail. At the top area, the supply line 24 for the body nozzle 28 is secured to the top rear of the housing 12, such as at a generally curved portion 12a. However, such a configuration is one of many configurations that can be used and still be with the scope of the present invention. The flexible tubing 24 is secured to the top rear of the housing 12 with the body nozzle 28, which is a substantially rigid needle-like member, extending laterally forward to meet with the top hole 34 to provide haptic air to the user's body, such as their face, lips, and the like.

In FIGS. 7 and 8 the supply line 26 for the mouth nozzle 30 can also clearly be seen secured to the lower portion of the rear of the housing 12 with its own connector 32. The mouth nozzle 30 is connected to and is in fluid communication with its respective supply line tube 26. The mouth nozzle 30 is also preferably rigid and is of a length so the free end thereof is located proximal to the edge 16a of the opening of the mouth port 16. While not shown, the nozzles 28, 30 can be adjustable so the haptic air flow therefrom can be directed, as desired by the user.

In general, as can best be seen in FIG. 8, the housing 12 is a hollow member with an open mouth port 16 and an open pass-through breathing channel 22 at the bottom. The mouth nozzle 30 and the body nozzle 28 are aiming in the same direction, namely, toward the user to effectively deliver the haptic air flow. When the user places the apparatus 10 against their lips, with the pass-through breathing channel 22 facing downward, the mouth nozzle 30 and body nozzle 28 are optimally oriented to deliver the desired haptic air flow to the user.

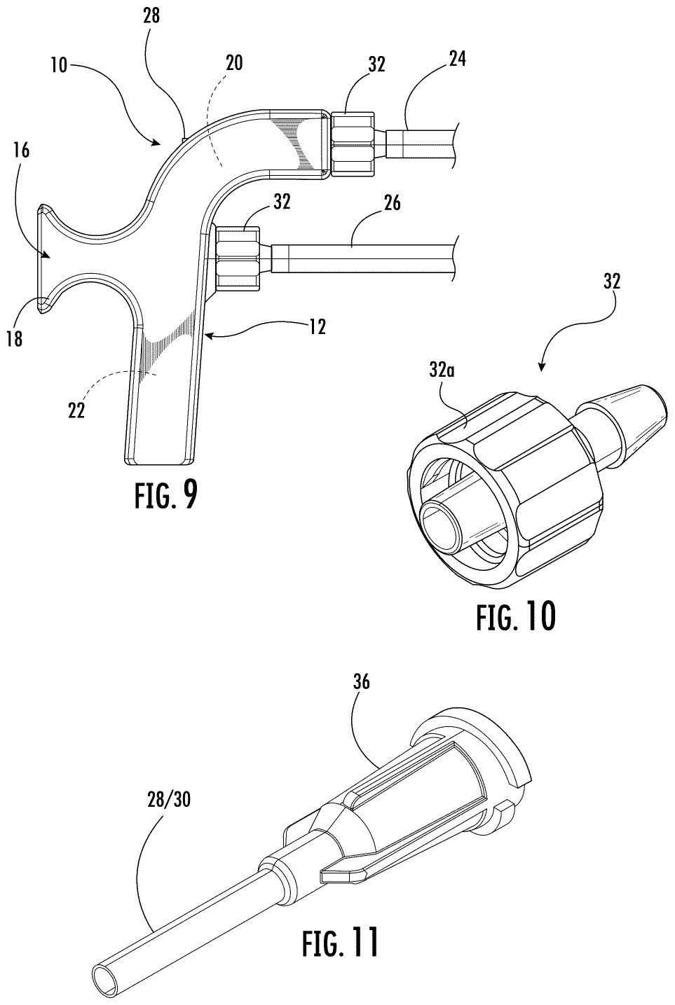

Turning now to FIGS. 9-11 details of the preferred embodiment of the interconnection of the supply line tubing to the housing 16 and nozzles 28, 30 is shown. FIG. 9 shows a side view of the housing 12 of the apparatus 10 where the supply lines 24, 26 laterally enter the housing 12 by the connectors 32 shown in FIG. 10. For example, the connectors 32 preferably have an outer threaded collar that threadably secure to a nozzle adapter end 36, as in FIG. 11, which is fixed to the rigid nozzle portion 28, 30. The rigid nozzle portion 28, 30 may be made of plastic, metal or other material. The adapter end 36 and threaded collar 32a of the connector 32 made secure to each other via complementary helical threading, luer lock, bayonet connection, or the like. Thus, the flexible air supply line tubing 24, 26 is adapted respectively to a rigid nozzle member 28, 30. The tubing 24, 26 is press or friction-fit on the to the opposing end of the connector 32. For assembly, the rigid nozzle portion 28, 30 is inserted into its respective hole 38, 40 in the rear of the housing 12 so the adapter end 36 of the nozzle 28, 30 sits within and in press-fit connection with the housing 12 at that location. FIG. 8 illustrates this preferred assembly of the apparatus 10 of the present invention. This construction greatly facilitates and speeds up install of the nozzles 28, 30 into the housing 12 and also enables them to be removed for cleaning, replacement, and the like. Also, this construction permits different nozzles 28, 30 to be installed, such as of different sizes, to produce a different air velocity.

It should be noted that the above connector 32 and interface between the air supply tubing line 24, 26 is just one of many different ways the supply lines 24, 26 can be fluidly connected to their respective nozzles 28, 30 and installed in place in the housing 12.

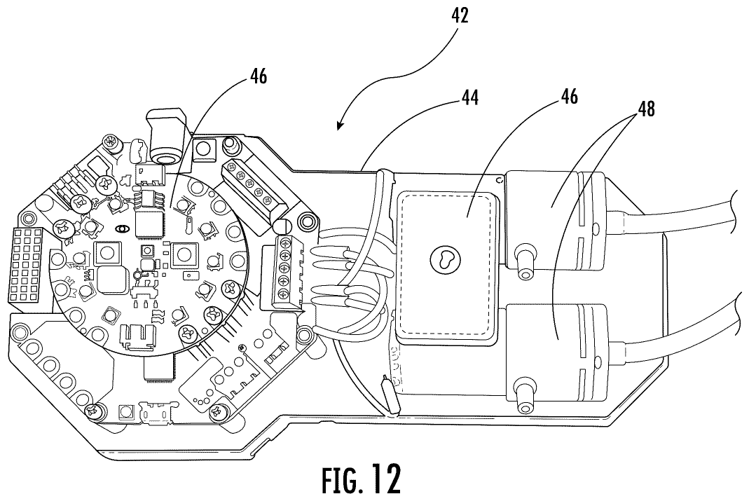

FIG. 12 illustrates an embodiment of a controller 42 having a circuit board 44, with components 46 thereon, for control of the apparatus 10 of the present invention. For illustration purposes, this controller 42 is shown without its housing so the components 46 therein can be easily seen in detail. For example, the controller 42 includes two pumps 48, one for supplying haptic air to the mouth nozzle 30 and the other for supplying haptic air to the body nozzle 28. The pumps may be of any type including a piezo disc pump, or the like. One pump may be used instead with flow controlled to the nozzles by a valve, such as an electronically controlled solenoid valve.

Also included on the circuit board 44, are antennas, vibrational motors, a preferably rechargeable battery for supplying power to the pumps, a charging port for charging (such as USB, Lightning and the like) (not shown). Also, inductive charging, such as Qi charging may be provided in additional or instead of wired charging. Also, a single-use battery is also possible for user convenience and battery durability. The controller 42 may include a control panel directly thereon with buttons and display (not shown) or, more preferably, a wireless communication module, that includes Bluetooth, Wi-Fi or other wireless protocol so the controller may communication wirelessly with other devices so those devices can control the controller 42 to, in turn, control the breathing assistance apparatus 10 of the present invention. It should be particularly understood that the controller 42 may be configured in any preferred fashion so it controls the operation of the haptic air pumps 48, as desired, to in turn control the overall operation of the breathing assistance apparatus 10 of the present invention.

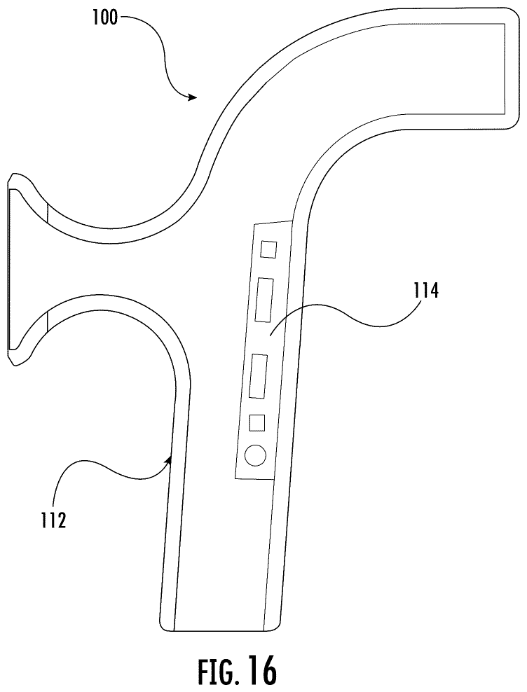

The present invention may also be provided in a fully integrated and entirely self-contained contained breathing assistance apparatus 100. FIG. 16 shows a cross-sectional representational view of the apparatus 100 where all of the components, such as the circuit board, pumps, tubing, and the like are miniaturized so that they may be fully enclosed within the body of the housing 112. FIG. 16 shows, generally and collectively as 114, all of the aforesaid operational components for apparatus 100 are all integrated into the housing 112 to provide an entirely self-contained compact apparatus 100. For ease of illustration, the nozzles are not shown in FIG. 16.

More specifically, all of the electronics and controller 114 are incorporated directly into the housing 100 of apparatus rather than having them as a separate component. The apparatus 100 in this embodiment includes all elements of the controller in the same housing 112 as the pump and air flow pathways (not shown) within member 114. The airflow ports may be integrated into a manifold rather or routed via internal tubing. These air flow pathways can be in the form of tubing as above or channels incorporated into the housing 112. Thus, an integrated and portable device 100 is provided which allows all functionality to be carried with a user. This integrated apparatus 100 may include various user interface elements as above, including nozzles for air to flow through towards the user, a mouthpiece for alignment with the user's mouth, coverings for sanitary storage, a charging port, indicator LEDs. Moreover, the member 114 may include other devices and components, such a vibratory device, which further provides vibratory haptic cueing to the user since the apparatus 100 is entirely handheld and being held by the user.

Also, in the embodiment of apparatus 100 in FIG. 16, the internal components 114 may modified to suit the fully enclosed configuration. For example, a single pump may be provided with a valve, which may be solenoid or otherwise, or, as above, two pumps may be provided that will direct the inhale-exhale air and cadence. In the integrated embodiment of FIG. 16, one or more pumps may be used to direct airflow to different locations. In one embodiment, airflow may be generated with one pump and directed to the appropriate nozzles via the valve. The valve may be mechanical, or electronically controlled, such as via a solenoid. In another embodiment, two or more pumps may be used, with each pump providing airflow in turn to the desired body locations to cue breathing.

The use of the apparatus 10 and 100 is shown in FIG. 13. It should be noted that the use of apparatus 10 and the integrated embodiment 100 are envision to be essentially the same. However, operation may be different depending on the components and functionality incorporated into the apparatus 10 and 100. An app 50 on a smartphone or tablet 52 can be used to connect to the controller 42 to, in turn, control the breathing assistance apparatus 10. FIG. 14 shows a computer laptop 54 that can also be connected to the controller 42 for the breathing assistance apparatus 10 to run an application program 56 thereon. Such a connection may be a wireless or wired connection. An application or website on such a remote device or computer can assist in the connection of that 52, 54 to the breathing assistance apparatus 10 wirelessly or by wired connection. The app 50 or application 56 can provide the user with selectable pre-set patterns, access educational resources about guided breathing and gather insight about their breathing practice.

On the remote device 52, 54, appropriate software is provided for the user to set up sessions with custom haptic feedback and timing as desired. The haptic air feedback maybe synchronized with music on the user's remote device 52, 54 to provide an enhanced session for the user. Sessions may be saved, shared, and the like. Thus, in general, using the companion mobile app 50 or application program 56 for user interaction reduces the need for complex and expensive user interface hardware on the apparatus 10 itself, aiding in ease of manufacture and cost reduction. Such a remote device 52, 54 and interconnection to a hardware medical device, like the breathing assistance apparatus 10 of the present invention, is so well known in the prior art that it need not be discussed in further detail herein.

For example, in use, the breathing assistance apparatus 10 of the present invention connects to a mobile app 50 or computer software 56, preferably using machine learning algorithms to collect user data and generate individualized breathing protocols. This personalized approach optimizes the effectiveness of the treatment. The companion app 50, 56 preferably has an artificial intelligence (AI) coach that the user interacts with for delivery of a breathing protocol that meets their current need and state and delivers educational materials to the user.

More specifically, as controlled by the controller 42 in the air pump portion 48 of the apparatus 10, air is supplied to the nozzles 28, 30 as desired and controlled by the companion application 50, 56. Thus, depending the programmed or set cadence, the delivery of haptic air “bursts” or “flow” are delivered appropriately to help cue the user when to breathe in, hold, and exhale. For example, the haptic air can be supplied into the user's mouth, to their upper lip or face and in any desired combination thereof. It is preferred that haptic air is delivered to different parts of the user's body depending on whether the cue is for inhale or exhale, for example. A common use would be for the user to inhale through the mouth and/or nose when a haptic air burst is felt in the mouth and then exhale through the mouth when a haptic air burst is felt on the body, such as on the face or lip. The present invention can accommodate any combination of inhale and exhale actions by the user. In general, the delivery of haptic air to the user is fully customizable by the user for real-time haptic feedback that mimics actual breathing patterns.

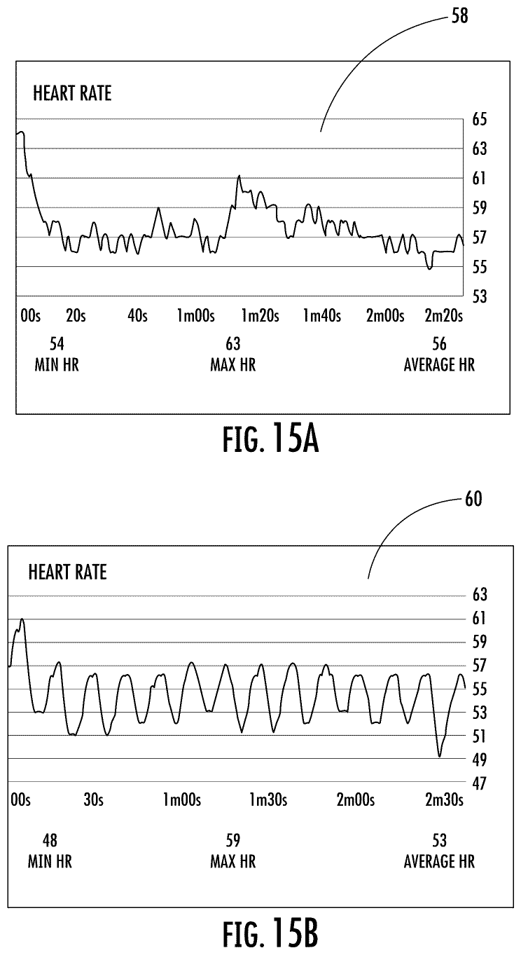

In actual use, one example goal would be to improve heart rate variability. When, an inhale haptic air event is be delivered into the mouth or on the upper lip of the user, this metric can be improved. Blowing air on the upper lip can also be used to induce or cue an exhale. It is also possible to blow haptic air on the nose, chin, sides of the mouth, or anywhere else on the user. All during this time, the pass-through channel 22 permits two-way breathing for inhale/exhale that allows the user to breathe in and out at a rate that is independent of the haptic air flow. With the regular use of the breathing assistance apparatus 10 of the present invention, such heart rate variability can be improved as seen in FIGS. 15A and 15B. FIG. 15A shows a graph 58 of variable heart rate of an individual before the user begins use of the breathing assistance apparatus 10 of the present invention. After regular use of the breathing assistance apparatus 10 of the present invention, such heart rate variability can dramatically improve, as seen in graph 60 in FIG. 15B, thereby providing a desirable calming, anxiety and stress reducing result.

Therefore, the apparatus 10 of the present invention addresses these limitations and shortcomings of prior art devices by providing a proactive, user-centered device and application solution that uses guided breathwork through airflow cueing, preferably, into the mouth and on the face allowing the users to follow along subconsciously with breathing patterns to prevent anxiety. Unlike pharmaceutical solutions, it comes with no risk of side effects or dependency. It provides a more tangible, interactive approach compared to digital-only meditation apps, and it goes beyond existing wearable tech by not only monitoring but also actively guiding the user's breathwork. Moreover, with its straightforward design and easy-to-source components, it is anticipated to be cost-effective, durable, and easy to manufacture.

The apparatus 10 of the present invention can be made of any type of material but the housing 12 and nozzles 28, 30 are preferably made of injection molded plastic while the tubing 24, 26 is made of silicone, nylon or other materials. The pumps 48 to supply the haptic air can be any type of pump and can deliver any type of air supply. For example, the haptic air could be very short air bursts or extended air flow, all of which can be configured in the controller 42 via the connected software application, which can be provided on any computing device, such a mobile phone, tablet or desktop computer.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A apparatus for assisting cued breathing, comprising:
a housing including at least one two-way air passage in communication with a mouth port; the two-way air passage being configured and arranged for communication with a user's mouth for two-way passage of breath air to and from the user's mouth;
a supply of air;
a first nozzle secured to the housing and connected to the supply of air via a first supply line;
wherein the first nozzle providing haptic air flow to deliver a haptic cueing event to a body portion of a user; and
wherein the haptic cueing event assists a user's breathwork cadence.

2. The apparatus of claim 1, further comprising:
a second nozzle secured to the housing and connected to a supply of air via a second supply line;
wherein the second nozzle providing haptic air flow to deliver a haptic cueing event to a body portion of a user.

3. The apparatus of claim 1, wherein the first nozzle is located in the mouth port of the housing and haptic air flow is delivered into a mouth of the user.

4. The apparatus of claim 2, wherein the second nozzle is located on the housing and in a different location than the first nozzle, the second nozzle delivering haptic air flow to a body part of the user other than inside the mouth.

5. The apparatus of claim 4, wherein the second nozzle delivers haptic air flow to the user's lip, face or chin.

6. The apparatus of claim 2, further comprising:
a first pump connected to the first supply line to provide air flow to the first nozzle; and
a second pump connected to the second supply line to provide air flow to the second nozzle.

7. The apparatus of claim 2, further comprising:
a pump selectively connected to the first supply line and the second supply line to provide air flow alternatingly to the first nozzle and the second nozzle.

8. The apparatus of claim 6, further comprising:
controller electronically connected to the first pump and the second pump for control thereof.

9. The apparatus of claim 7, further comprising:
controller electronically connected to the pump for control thereof.

10. The apparatus of claim 8, wherein the controller includes a wireless communication module for wirelessly communicating with a remote device.

11. The apparatus of claim 9, wherein the controller includes a wireless communication module for wirelessly communicating with a remote device.

12. The apparatus of claim 2, wherein the first supply line and the second supply line are made of silicone or nylon.

13. The apparatus of claim 2, wherein the first nozzle and the second nozzle are needles in fluid communication with their respective first supply line and second supply line.

14. The apparatus of claim 1, wherein the housing is made of plastic.

15. The apparatus of claim 8, wherein the controller is customizable to provide an any desired inhale/exhale breathwork session pattern and timing.
